(19) United States
(12) Patent Application Publication
(10) Pub. No.: US 2021/0298733 A1
(43) Pub. Date: Sep. 30, 2021

(54) DEVICES AND METHODS FOR VISUALIZATION OF A SPINAL SURGICAL SITE

(71) Applicant: Providence Medical Technology, Inc., Pleasanton, CA (US)

(72) Inventors: Shigeru Tanaka, Half Moon Bay, CA (US); Jeffrey D. Smith, Clayton, CA (US); Christopher U. Phan, Dublin, CA (US); Edward Liou, Pleasanton, CA (US); Christopher Lambert, Concord, CA (US); Nicholas Domek, Walnut Creek, CA (US)

(21) Appl. No.: 17/266,704

(22) PCT Filed: Aug. 9, 2019
(86) PCT No.: PCT/US2019/045875

§ 371 Date: Feb. 8, 2021

Related U.S. Application Data
(60) Provisional application No. 62/716,708, filed on Aug. 9, 2018.
(60) Provisional application No. 62/835,077, filed on Apr. 17, 2019.

(51) Int. Cl. A61B 17/02; A61B 17/34

(52) U.S. Cl. CPC A61B 17/025; A61B 17/0293; A61B 2017/0256; A61B 17/3439; A61B 17/3423

(57) ABSTRACT

Disclosed herein is an improved tissue retraction and direct visualization tool of a spinal surgical site, such as a site in the cervical spine, including a retractor assembly having a visualization channel, a first retractor member, and a second retractor member, the retractor assembly configured to be coupled to an actuator assembly configured to actuate the retractor assembly. In some aspects, the retractor assembly may transition from an unexpanded state for delivery to an expanded state for visualization. In some aspects, the retractor assembly is expanded with an actuator assembly. A method of using the visualization tool is also disclosed.

### CROSS REFERENCE TO RELATED APPLICATIONS

This application claims priority to U.S. Provisional Patent Application No. 62/716,708, filed Aug. 9, 2018 and U.S. Provisional Patent Application No. 62/835,077, filed Apr. 17, 2019, each of which is incorporated by reference herein in its entirety.

### TECHNICAL FIELD

The technology described herein relates generally to devices and methods directed to spinal surgical sites, and more specifically to methods and apparatus for tissue retraction and direct visualization of a spinal surgical site.

### BACKGROUND

In existing spinal fusion procedures, tissue must be retracted for direct visualization of the surgical site. The tissue may be retracted using a retractor instrument. These retractor instruments may be cumbersome and difficult to deploy, and the retractor instrument may be not be able to contact and retract all the surrounding tissue fully.

It is therefore desirable to provide an improved retractor device that addresses the above-described problems and/or which more generally offers improvements or an alternative to existing arrangements.

The information included in this Background section of the specification, including any references cited herein and any description or discussion thereof, is included for technical reference purposed only and is not to be regraded subject matter by which the scope of this disclosure as defined in the claims is to be bound.

### SUMMARY

Disclosed herein is an improved tissue retraction and direct visualization tool of a cervical spine surgical site. In one example, the visualization device includes a retractor assembly having a visualization channel, a first retractor member, and a second retractor member, the retractor assembly configured to be coupled to an actuator assembly configured to actuate the retractor assembly.

In one example, the retractor assembly is expandable.

In one example, the first retractor member is expandable.

In one example, the first retractor member is expanded and configured to cause the second retractor member to expand.

In one example, the first retractor member is radially expandable.

In one example, the first retractor member is configured to move with respect to the second retractor member.

In one example, the second retractor member is configured to move with respect to the first retractor member.

In one example, the first retractor member and the second retractor member are configured to move with respect to each other.

In one example, the first retractor member comprises a mesh structure formed into an expandable spring or spring-like configuration having looped spring coils stacked between a proximal end and a distal end, and the visualization channel is formed by an inside surface of the coil springs.

In one example, the first retractor member is positioned within the second retractor member.

In one example, the second retractor member is a sheath.

In one example, the first retractor member forms the visualization channel.

In one example, a surface of the first retractor member forms the visualization channel.

In one example, the first retractor member comprises radially expanding blades.

In one example, the actuator assembly is configured to be rotated with respect to the retractor assembly.

In one example, the retractor assembly is toroidally expandable.

In one example, the device includes the actuator assembly.

In one example, the surgical site is in the cervical spine.

In one example, a distal end of the retractor assembly is tapered.

In one example, the retractor assembly has an adjustable diameter.

In another example, the visualization device includes a retractor assembly having a visualization channel that extends from a proximal end to a distal end of the retractor assembly, and a tool portal adjacent the distal end.

In one example, the surgical site is in the cervical spine.

In one example, the device includes a visualization aid positioned adjacent a proximal end of the retractor assembly.

In one example, the tool portal extends a length of the retractor assembly between a proximal end and the distal end.

In one example, the retractor assembly comprises a sleeve with an adjustable diameter.

In another example, a method of using a visualization device in a surgical site includes inserting at least a portion of a retractor assembly into an incision and positioning adjacent the surgical site, engaging an actuator assembly with the retractor assembly, expanding the retractor assembly, and expanding a visualization channel of the retractor assembly extending from a proximal end to a distal end of the retractor assembly.

In one example, the method includes flowing a fluid from the actuator assembly and into the retractor assembly.

In one example, the method includes fluidly connecting the actuator assembly and the retractor assembly.

In one example, the method includes unrolling the retractor assembly in an expanded direction towards the surgical site

In one example, the method includes further comprising distracting and retracting tissue about the retractor assembly.

In one example, the method includes disengaging the actuator assembly from the retractor assembly.

In one example, the method includes radially expanding the retractor assembly.

In one example, the method includes rotating the actuator assembly.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present disclosure as defined in the claims is provided in the following written description of various embodiments of the disclosure and illustrated in the accompanying drawings.

### BRIEF DESCRIPTION OF THE DRAWINGS

The accompanying drawings, which are incorporated into and constitute part of the specification, illustrate embodiments of the disclosure and, together with the general description above and the detailed description below, serve to explain the principles of these embodiments.

FIG. 1 is a perspective view of a visualization device in an unexpanded state, according to aspects of the present disclosure;

FIGS. 1A and 1B are exemplary views highlighting the spine, which may form a portion of the surgical environment, according to aspects of the disclosure;

FIG. 2 is a top view of the device of FIG. 1 as positioned in an example surgical environment;

FIG. 3 is a side view of the device of FIG. 2;

FIG. 4 is a perspective view of the device of FIG. 1 in an expanded state;

FIG. 5 is a top view of the device of FIG. 4;

FIG. 6 is a perspective view of a visualization device in an expanded state, according to aspects of the present disclosure;

FIG. 7 is a top view of the device of FIG. 6;

FIG. 8 is a perspective view of another visualization device in an unexpanded state, according to aspects of the present disclosure;

FIG. 9 is a top view of the device of FIG. 8 as positioned in an example surgical environment;

FIG. 10 is a side view of the device of FIG. 9;

FIG. 11 is a perspective view of the device of FIG. 8 in an expanded state;

FIG. 12 is a side view of the device of FIG. 11;

FIG. 13 is a top view of the device of FIG. 11;

FIG. 14 is a rear perspective view of the device of FIG. 11;

FIG. 15 is an exploded perspective view of a visualization device according to aspects of the present disclosure;

FIG. 16A is a side view of the visualization device of FIG. 15 as positioned in an example surgical environment in an unexpanded and expanded state;

FIG. 16B is a side view of an alternate embodiment of the visualization device of FIG. 15 as positioned in an example surgical environment in an unexpanded and expanded state;

FIG. 17 is a side view of the visualization device of FIG. 15 as positioned in an example surgical environment;

FIG. 18 is a top view of the visualization device of FIG. 17;

FIG. 19 is a side view of the visualization device of FIG. 17 where the outer sheath is removed;

FIG. 20 is a side view of a visualization device in an unexpanded and expanded state, according to aspects of the present disclosure;

FIG. 21 is a side view of a visualization device in different orientations, and in an expanded state as positioned in an example surgical environment, according to aspects of the present disclosure;

FIG. 22 is a side view of a portion of a visualization device which may be used with other visualization devices, according to aspects of the present disclosure;

FIG. 23 is a perspective view of a visualization device, according to aspects of the present disclosure;

FIG. 24 is a perspective view of the device of FIG. 23 in an expanded states;

FIG. 25 is a top view of the device of FIG. 24;

FIG. 26 is an exploded, perspective view of the visualization device of FIG. 23, shown with an actuator assembly;

FIG. 27 is a side view of the visualization device of FIG. 26 as positioned in an example surgical environment;

FIG. 28 is a top view of the visualization device of FIG. 27;

FIG. 29 is a perspective view of the visualization device of FIG. 26 in use;

FIG. 30 is a partial top view of a visualization device, according to aspects of the present disclosure;

FIG. 31 is a partial top view of the device of FIG. 30 in an expanded state;

FIG. 32 is a perspective view of the device of FIG. 30 positioned in an example surgical environment;

FIG. 33 is a top view of FIG. 32;

FIG. 34 is a side view FIG. 32;

FIG. 35 is a partial exploded perspective view of a visualization device, according to aspects of the present disclosure;

FIG. 36 is an exploded perspective view of the visualization device of FIG. 35;

FIG. 37 is a perspective view of a component of the visualization device of FIG. 35;

FIG. 38 is a side view of a portion of the visualization device of FIG. 35;

FIG. 39 is a partial side view of a visualization device, according to aspects of the present disclosure;

FIG. 40 is a perspective view of a visualization device, according to aspects of the present disclosure;

FIG. 41 is a top view of the device of FIG. 40;

FIG. 42 is a perspective view of the device of FIG. 40 in an expanded state;

FIG. 43 is a perspective view of the device of FIG. 40 positioned in an example surgical environment;

FIG. 44 is a top view of FIG. 40;

FIG. 45 is a top view of FIG. 42, wherein the device is expanded by rotation;

FIG. 46 is a top view of FIG. 42, wherein the device is expanded by outward pressure;

FIG. 47 is a perspective view of a visualization device as positioned in an example surgical environment, according to aspects of the present disclosure;

FIG. 48 is a perspective view of the visualization device of FIG. 47, including a surgical tool;

FIG. 49 is a top view of FIG. 48 as positioned in an example surgical environment;

FIG. 50 is a perspective view of a visualization device as positioned in an example surgical environment, according to aspects of the present disclosure;

FIG. 51 is a cross-sectional side view the visualization device of FIG. 50, shown with a surgical tool;

FIG. 52 is a perspective view of FIG. 51 wherein the surgical tool is shown in a different position;

FIG. 53 is a cross-sectional side view of FIG. 52 as positioned in an example surgical environment;

FIG. 54 is an exploded, perspective view of a visualization device, according to aspects of the present disclosure;

FIG. 55 is a perspective view of the visualization device of FIG. 54;

FIG. 56 is a perspective view of the visualization device of FIG. 55 in an expanded state;

FIG. 57 is a perspective view of the visualization device of FIG. 15.

### DETAILED DESCRIPTION

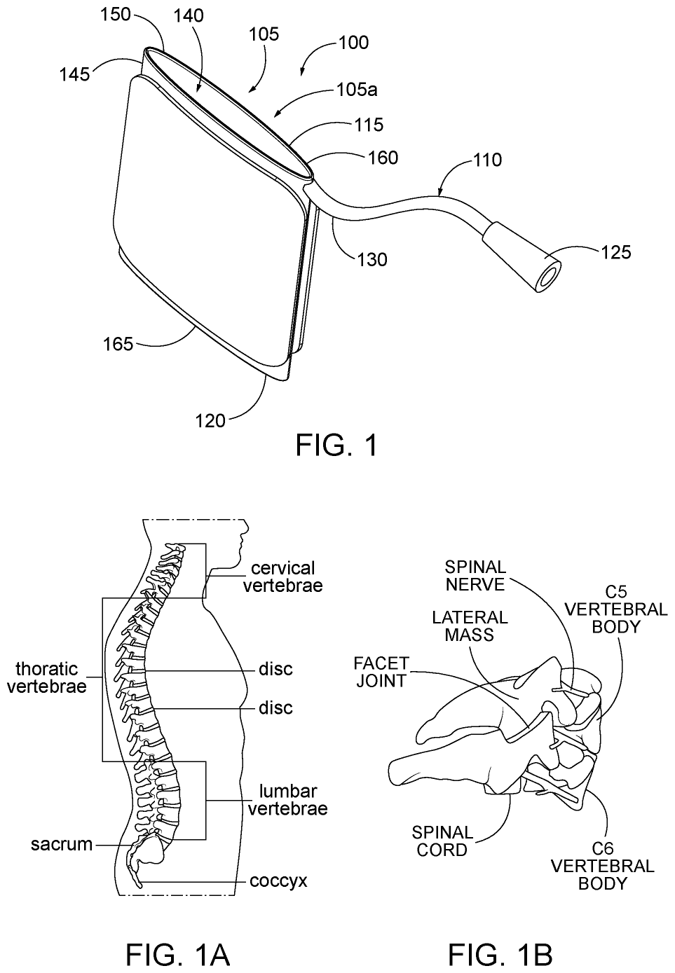

The various embodiments described herein relate to devices for the retraction of tissue to create direct visualization of a surgical site during surgery. Examples and embodiments may include a visualization device having a retractor assembly and an actuator assembly. In some examples, the visualization device is used to provide a user direct visualization of a surgical site during surgery. In some examples, the visualization device is expandable. In some examples, the actuator assembly is used to actuate the visualization device, such as to be inserted into, coupled with, connected to, or expand the visualization device in, around, or target a targeted surgical site. In various embodiments, the target surgical site is the spine, such as the cervical spine, as shown in FIGS. 1A and 1B. In various embodiments, the target site is a cervical facet joint. In some examples, the visualization device may be included or used with a system of instruments for access, bone preparation and decortication, bone graft application, and implant delivery. In some examples, the visualization device may be used in a system for anterior cervical discectomy and fusion (ACDF).

There are multiple examples and embodiments of methods to actuating the visualization device. In an example, an incision is made in the posterior cervical spine (see FIGS. 1A and 1B for reference), and a manual dissection of tissue is performed down to the surgical area. A distal end of the retractor assembly of the visualization device is positioned within the incision and into the area where the tissue has been manually dissected and positioned adjacent the surgical area. An actuator assembly may then be engaged to actuate the expansion of the retractor assembly from an unexpanded position or state to an expanded position or state to form or increase the size of a visualization channel. This expansion may aid a user, such as a health care professional, to better visualize the surgical site and operate instruments within the visualization channel.

Turning now to the figures, a visualization device is disclosed which may be used to help visualize and/or access a surgical site, such as a surgical site in the spine.

FIGS. 1-5 include various views of a visualization device. FIG. 1 depicts a visualization device 100 with a retractor assembly 105 and an actuator assembly 110. In FIG. 1, the retractor assembly 105 is shown in an unexpanded state or position. The retractor assembly 105 has a proximal end 160, a distal end 165, and first retractor member 115. In some examples, the retractor assembly 105 may be ring shaped having a first or outside surface 145 and a second or inside surface 150 that help form a visualization channel 140 defined by the first retractor member 115.

The retractor assembly 105 may also include a second retractor member 120, which may be at least one retractor plate. In some examples, the retractor assembly 105 may have more than one first retractor member 115, and/or more than one second retractor member 120 that combine to form the retractor assembly 105.

In some examples, the second retractor members 120 are positioned on the outside surface 145 of the first retractor member 115. In some examples, the second retractor member 120 may be rigidly attached to the outside surface 145. In some examples, the second retractor members 120 may be attached to the outside surface 145 of the first retractor member 115 in a manner that allows the second retractor members 120 to be mobile or articulating with respect to the first retractor member 115. In some examples, the second retractor member 120 may add to the overall strength of rigidity of the retractor assembly 105. In some examples, the first retractor member 115 provides structure and may act as a retractor element against tissue such that a second retractor member, such as retractor plates, are not used or needed.

In some examples, the retractor plates are placed on the inside surface of the first retractor member. In these examples, the first retractor member may contact the surrounding tissue. In some examples, the contact of the first retractor member with the surrounding tissue may be less traumatic than in an example where a retractor plate contacts the surrounding tissue. In some examples, the ability of a visualization device with the retractor plates positioned inside the first retractor member may reduce the ability of the visualization device to retract effectively due to material compliance. A retractor plate placed within the first retractor member would help make the visualization channel more visible, and also help protect the first retractor member from potential damage from contact with instruments used within the visualization channel.

In some examples, the retractor assembly 105 may be expandable. In some examples, the first retractor member 115 is expandable, and in other examples, the second retractor member 120 is expandable. In some examples, the first retractor member 115 may be expanded in a relatively similar manner about an overall circumference of the first retractor member 115, such that the first retractor member 115 is evenly or uniformly expanded. In some examples, the expandable first retractor member 115 may be expanded by inflation. The expandable retractor member 115 may be flexible or pliable, so that its form factor may be different in an unexpanded state compared to an expanded state. The retractor assembly 105 of FIG. 1 is shown in an unexpanded state 107a. In some examples, in the unexpanded state or position 107a, the first retractor member 115 is deflated or partially inflated. In some examples, in the unexpanded state 107a, the retractor assembly 105 may have a flattened, smaller profile that may aid in the introduction of the retractor assembly 105 into a surgical incision.

In some examples, the retractor assembly 105 may include a visualization channel 140. In the example of FIG. 1, the first retractor member 115 may be a ring or have a circular shape with an opening in the center, formed by the second surface 150, that forms the visualization channel 140. In some examples, the retractor assembly may include multiple visualization channels.

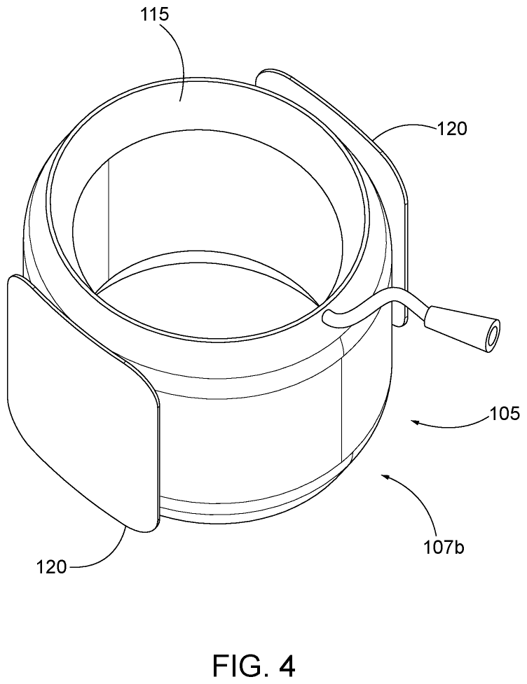

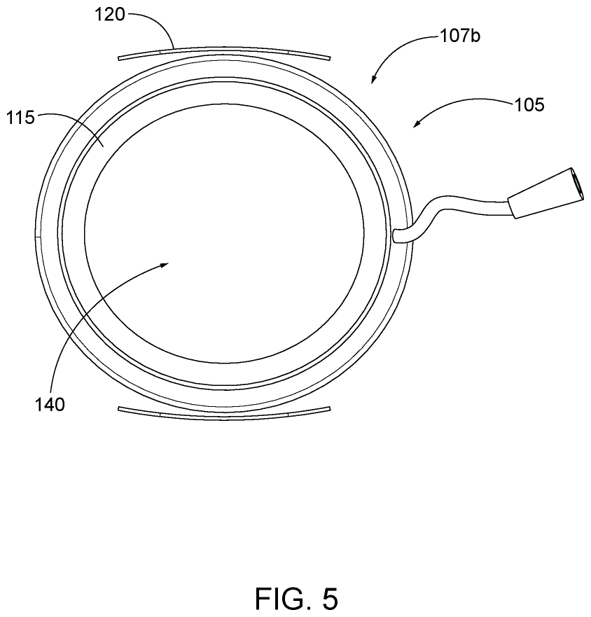

FIGS. 4 and 5 show the visualization device 100 of FIG. 1 in an expanded position 107b. In the example of FIGS. 4 and 5, the first retractor member 115 is expanded. In the expanded position 107b, the opening forming the visualization channel 140 of the retractor assembly 105 is enlarged. In some examples, the enlarged visualization channel 140 may create a visualization path that helps a user to see through the visualization channel 140 to better visualize the surgical site and operate instruments within it.

The visualization device 100 of FIG. 1 may also include an actuator assembly 110. In some examples, the actuator assembly 110 may include a first actuator member 125 and a second actuator member 130. In the example of FIG. 1, the first actuator member 125 may include an inflation tube and the second actuator member 130 may include a port. In some examples, the first actuator member 125 and second actuator member 130 may be fluidly coupled to the retractor assembly 105 near its proximal end 160. In some examples, the first actuator member 125 and second actuator member 130 may be fluidly connected to the first retractor member 115. In operation, a fluid or gas may be injected or inserted into the first actuator member 125 and flow through past the second actuator member 130. In some examples, such as where the second actuator member 130 is a port, the second actuator member 130 may be engaged to allow or prevent the supplied fluid from flowing through the second actuator member 130, or control the volume of flow through the second actuator member 130 and ultimately into or out of the retractor assembly 105.

In other examples, the retractor assembly 105 of the visualization device 100 may be engaged with an actuator assembly 110 and may include additional components that may be used to expand the visualization device 100 or to help align or place the visualization device 100 into a surgical environment.

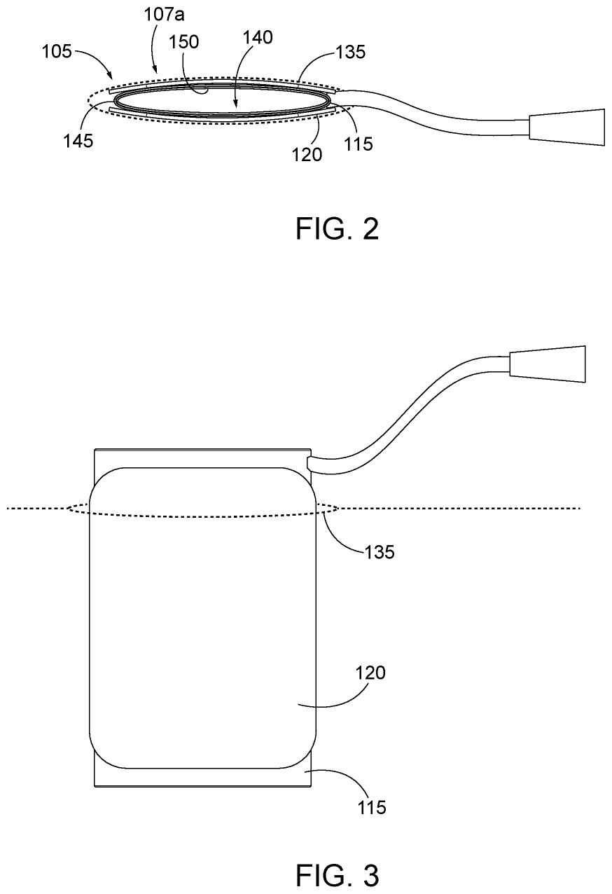

In an example, FIGS. 2 and 3 show the visualization device 100 of FIG. 1 as positioned in an example surgical environment. In some use examples, an incision 135 is made in the posterior cervical spine, and a manual dissection of tissue is performed down to the surgical area. In some examples, the incision 135 may be linear. In other examples, the incision 135 may be non-linear. In some examples, the distal end 165 of the retractor assembly 105 of the visualization device 100 is then positioned within the incision 135 and into the area where the tissue has been manually dissected and positioned adjacent the surgical area. As shown in FIG. 2, a circumference of the incision 135 may be larger than an outer circumference of a portion of the retractor assembly 105. This may allow a portion of the retractor assembly 105 to be inserted into or positioned partially within the incision 135 without creating additional damage to the skin area adjacent the incision. In some examples the second retractor member 120, such as multiple retractor plates, may be aligned with the incision 135.

In operation, the actuator assembly 110 may be engaged to actuate the expansion of the retractor assembly 105 from an unexpanded position 107a (FIG. 1) to an expanded position 107b (FIG. 4). In some examples, a fluid supply may be connected to the actuator assembly 110 to allow fluid to flow through the actuator assembly 110 and into the retractor assembly 105, such as into the first retractor member 115. This fluid flow may cause the first retractor member 115 to begin to expand and change shape or size and form the ring shape shown in FIG. 4. The expansion of the retractor assembly 105 may cause the second retractor member 120, such as retractor plates, to push against the tissue between the incision 135 and the surgical site. The expansion of the first retractor member 115 changes the shape of the visualization channel 140 so that it is expanded or opened. The expanded or open visualization channel 140 may allow a user, such as a health care professional, to better visualize the surgical site and operate instrument within the visualization channel 140.

In examples where the second retractor member 120 is formed as a retractor plates(s), the second retractor member 120 may provide additional structure to help strengthen the overall retractor assembly 105, maintain the visualization channel 140, and prevent the retractor assembly 105 from unintentionally collapsing or transitioning back to an unexpanded position. The second retractor member 120 may help evenly disperse any forces acting inward or onto the retractor assembly 105 from the displaced tissue surrounding the retractor assembly 105.

In some examples, the first retractor member 115 is capable of providing the appropriate structure to act as a retractor element against the displaced tissue, and a second retractor member, such as a retractor plate, may be omitted.

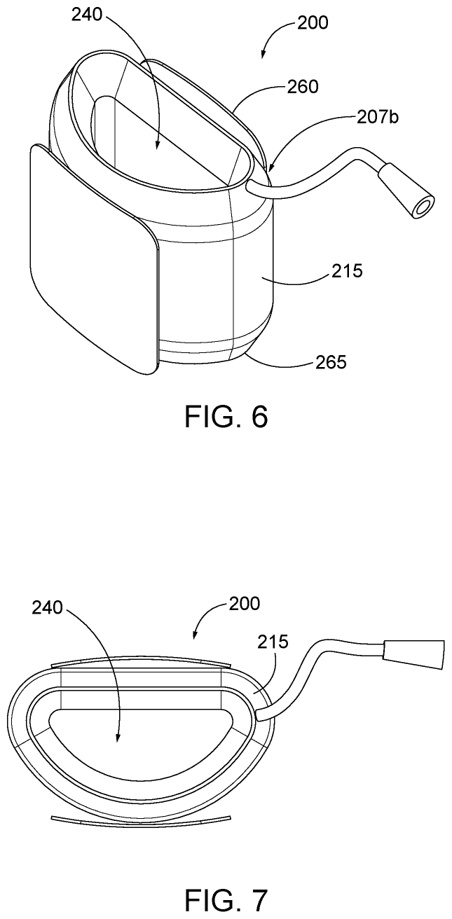

Turning now to FIGS. 6 and 7, the visualization device 100 may be expanded in a different way. The visualization device 200 may be similar to the described visualization device 100 of FIGS. 1-5. In some embodiments, a difference may be in that the first retractor member 215 may be expanded in a biased manner, so that only a portion of the first retractor member 215 expands, or so that the expansion creates a visualization channel 240 with a specific shape in an expanded position 207b. In some examples, the first retractor member 215 may be expanded to form a “D” like shape. In some examples, the first retractor member 215 may be biased about a width of the first retractor member 215. In some examples, the first retractor member 215 may be biased about a length of the retractor assembly 205 between a proximal end 260 and a distal end 265. In some examples, the visualization device 200 may have other expandable components that are biased.

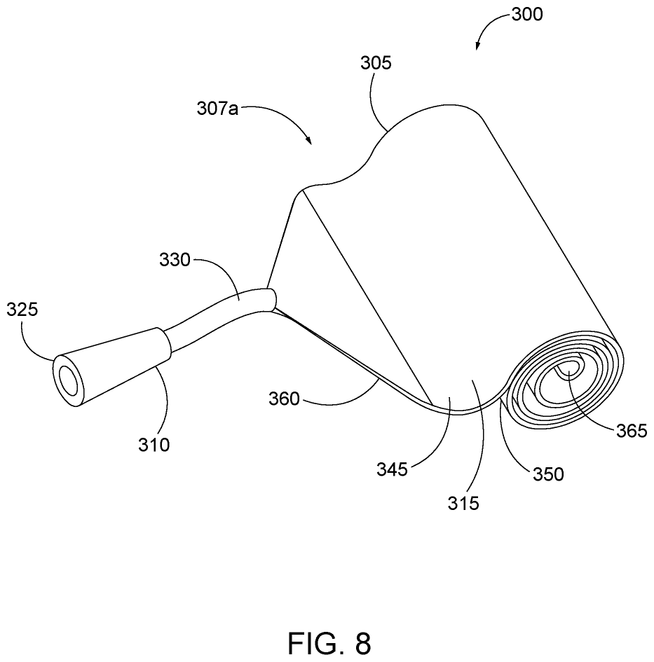

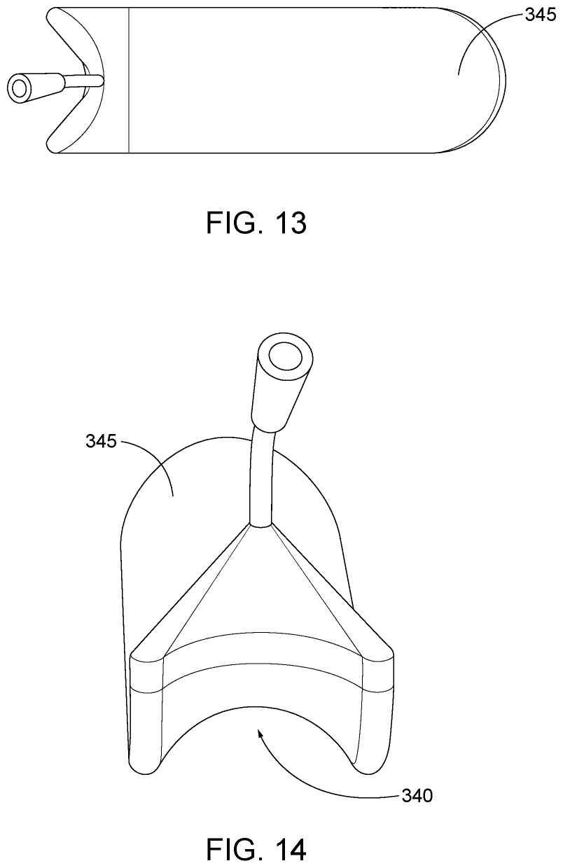

The visualization device 300 of FIGS. 8-14 may be similar to the visualization devices 100, 200 of FIGS. 1-7, and include a retractor assembly 305 and an actuator assembly 310.

In the examples of FIGS. 8-10, the visualization device 300 includes a retractor assembly 305 in an unexpanded state or position 307a. The retractor assembly 305 has a proximal end 360 and a distal end 365. The retractor assembly 305 may include a first retractor member 315 positioned in an unexpanded configuration such that the distal end 365 of the retractor assembly 305 is rolled or folded within the first retractor member 315 in the unexpanded position 307a. The first retractor member 315 may have a first surface 345 and a second surface 350.

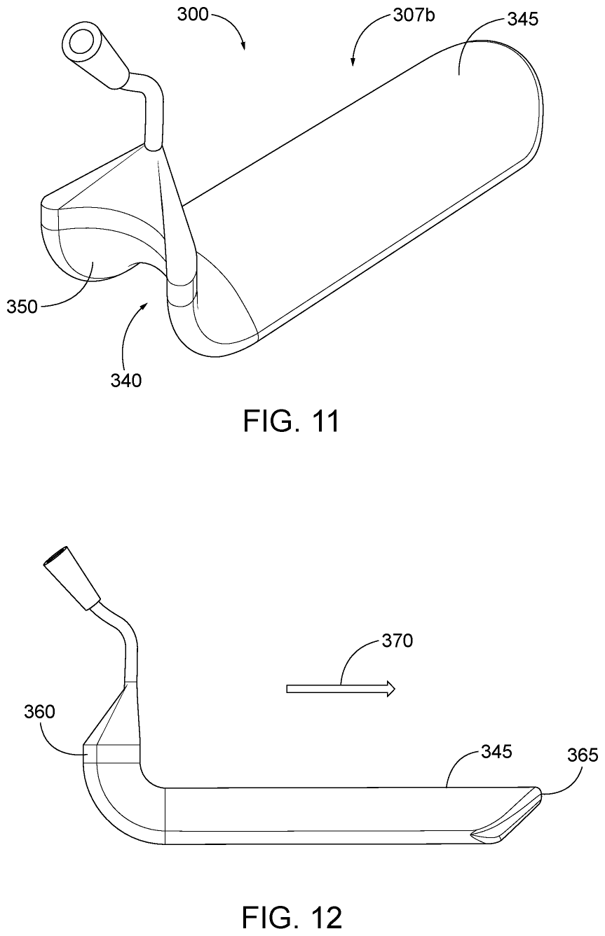

FIGS. 11-14 show the visualization device 300 in an expanded position 307b. In an example, the visualization device 300 may form an elongated U-shape, with the visualization channel 340 formed by the curved shape of the second surface 350 and the proximal end 360 adjacent the actuator assembly 310 and the distal end 365 opposite the proximal end 360.

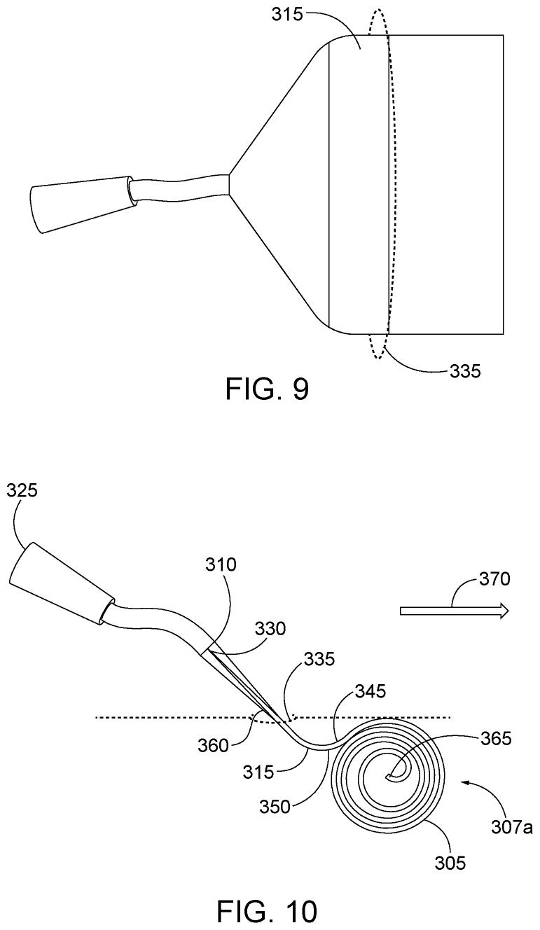

In an example, FIGS. 9 and 10 show the visualization device 300 of FIG. 8 as positioned in an example surgical environment. Similar to the visualization device 100 of FIGS. 2 and 3, an incision 335 may be made in the posterior cervical spine and some manual dissection may be performed to the surgical site. The distal end 365 of the unexpanded retractor assembly 305 of the visualization device 300 may then be inserted into the incision 335, with the expanded direction 370 aligned with the anatomical space that should be retracted. Once placed in the incision 335, the retractor assembly 305 may then be expanded. During the transition from the unexpanded position 307a to the expanded position 307b, in an example where the retractor assembly 305 is expanded via inflation, the retractor assembly 305 unrolls forward in the expanded direction 370 and expands into an elongated U-shape, dissecting and retracting tissue along its path. In some examples, unlike the visualization device 100, the amount of manual dissection required may be less when using the visualization device 300, since during the transition from the unexpanded position 307a to the expanded position 307b, the retractor assembly 305 will also dissect the tissue near the surgical site.

Once transitioned to an expanded position 307b, the expanded retractor assembly 305 creates a visualization channel 340 from the proximal end 360 to the distal end 365 with an elongated space defined by the U-shape of the retractor assembly 305 and its second surface 350. Similar to the first and second embodiments 100, 200, in use, the health care professional is able to visualize the surgical site through the visualization channel 340, and utilize instruments through and within the visualization channel 340.

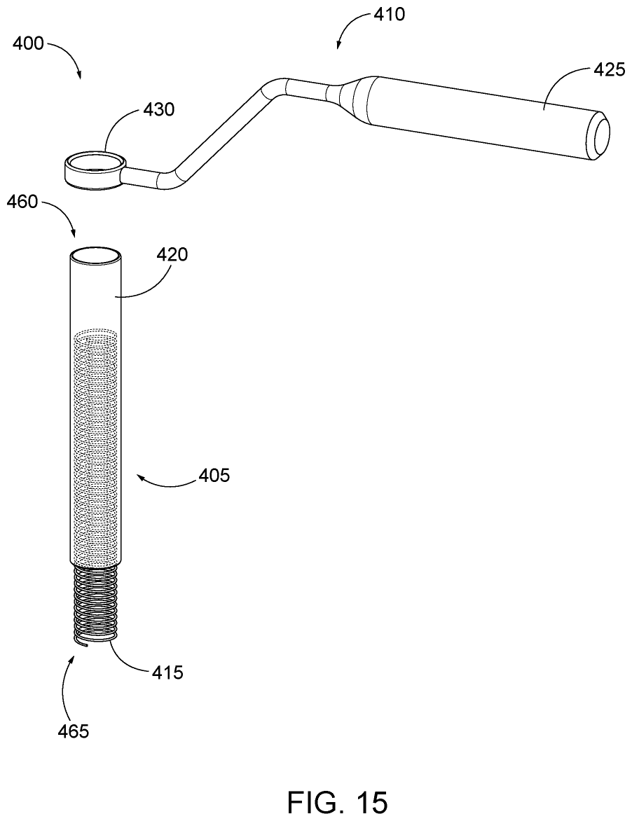

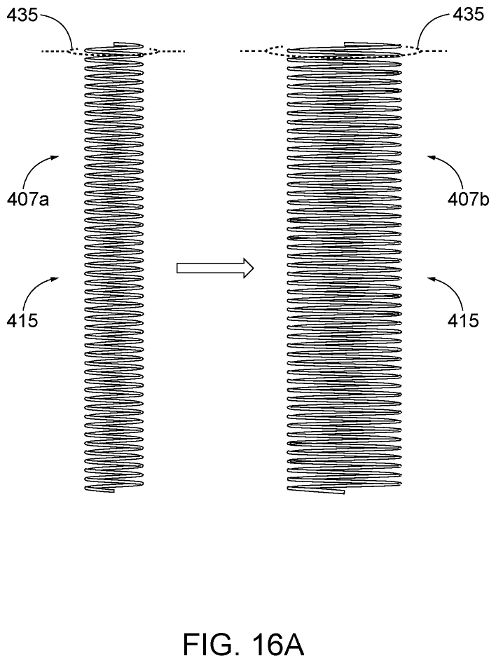

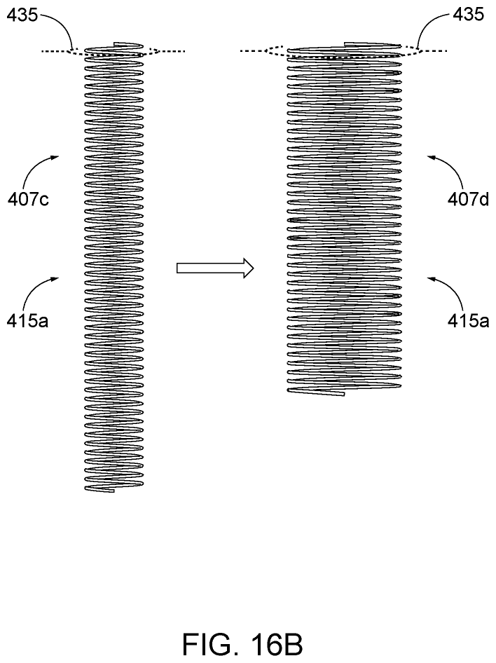

FIGS. 15-19 show a visualization device 400 with a retractor assembly 405 having a proximal end 460 and a distal end 465, an actuator assembly 410, a first retractor member 415, and a second retractor member 420. In some examples, the first retractor member 415 may be a mesh structure formed into an expandable spiral or spring like configuration with looped spring coils stacked between the proximal end 460 and the distal end 465 and a visualization channel 440 formed by an inside surface of the spring coils. In some examples, the second retractor member 420 may be formed as an outer sheath. FIG. 16A includes the first retractor 415 of the retractor assembly 405 in an unexpanded position 407a and the first retractor 415 of the retractor assembly 405 in an expanded position 407b. In some examples, the second retractor member 420 has an inside diameter that is larger than an outer diameter of the first retractor member 415. This may enable the second retractor member to be slid over or positioned about the first retractor member 415. In some examples, the first retractor member 415 and the second retractor member 420 have similar lengths.

FIG. 16B includes a first retractor 415a, which may be similar in some aspects to the first retractor 415 of FIG. 16A. The height of the retractor 415a in an unexpanded position 407c is longer than that of a height of the retractor 415a in the expanded position 407d. The overall length of the coiled material used to form the retractor 415a remains unchanged between the unexpanded position 407c and the expanded position 407d.

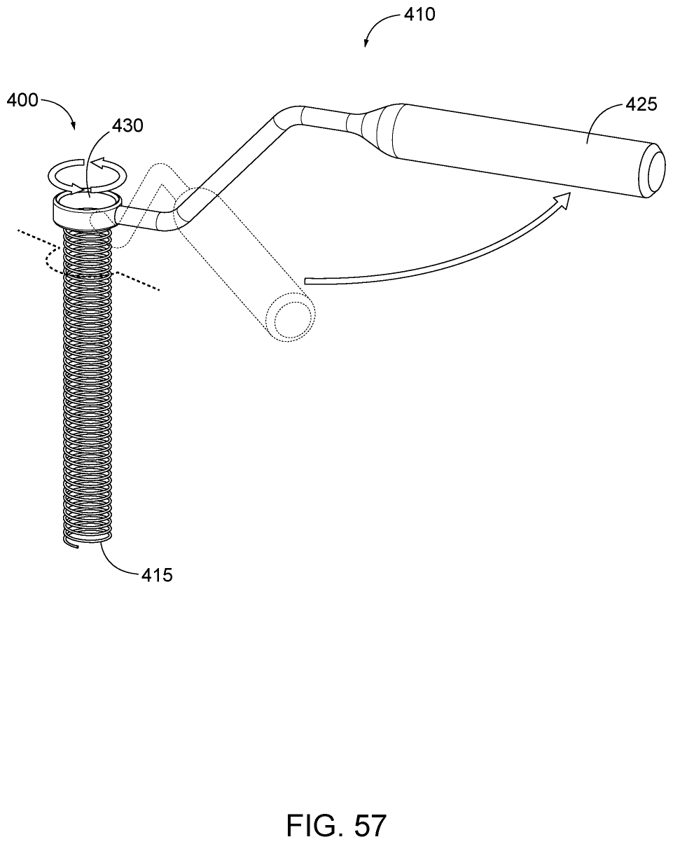

The device may further include an actuator assembly 410 shown in FIGS. 15 and 57. The actuator assembly 410 may include a first actuator member 425 and a second actuator assembly 430. In some examples, the first actuator member 425 may include a handle, and the second actuator member 430 may include a loop that may couple with the retractor assembly 405. In some examples, the second actuator member 430 may couple with the retractor assembly 405 using a mechanism that is a friction fit, a bayonet-type connection, a secondary fastener (such as a set screw or other fastener), a quick-connect type mechanism, a push-type mechanism, or other fully releasable coupling mechanisms. In some examples, the second actuator member 430 may engage with an outer surface of the retractor assembly 405 using a friction fit. In some examples, the second actuator member 430 may engage with an inner surface of the retractor assembly 405 using a friction fit. In some examples, the retractor assembly 405 may have features positioned adjacent to or that extend away from a surface of the retractor assembly 405 that engage with apertures, cutouts, detents or similar features of the second actuator member 430 or the actuator assembly 410. In some examples, the retractor assembly 405 may have apertures, cutouts, detents or similar features that engage with features formed on a surface or extending away from a surface of the of the second actuator member 430 or the actuator assembly 410. In some examples, the actuator assembly and retractor assembly may use a variety or combination of coupling or connection mechanisms, such as a combination of those disclosed herein.

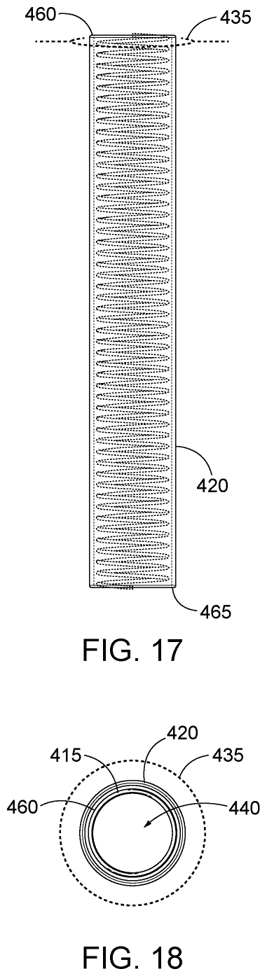

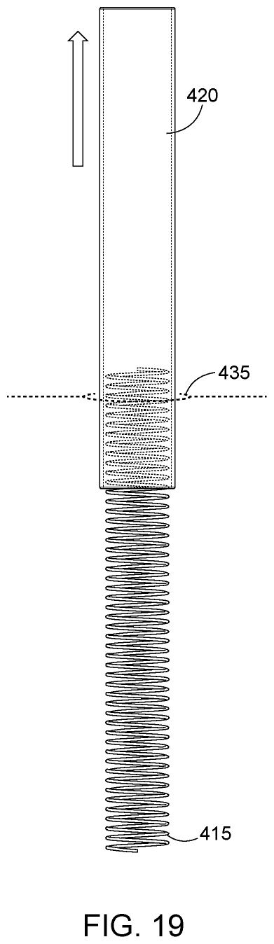

In an example use, an incision 435 is made in the posterior cervical spine, and a manual dissection is performed down to the surgical area. As shown in FIGS. 17 and 18, the distal end 465 of the retractor assembly 405, in its unexpanded position 407a, is inserted into the incision 435. As the retractor assembly 405 is inserted into the incision, the second retractor member 420, such as an outer sheath, may further retract, displace, or dissect the tissue near the surgical area. The second retractor member 420 may act to protect the first retractor member 415, such as spiral mesh, from catching or engaging with tissue near the surgical area prior to when the first retractor member 415 is expanded. This may help to prevent unnecessary damage to the surrounding tissue. Once placed within the incision 435, the second retractor member 420 may be separated or removed from the first retractor member 415 and removed from the incision 435, as shown in FIG. 19. As shown in FIG. 57, the actuator assembly 410, such as the spiral mesh actuator, may then be rotated with respect to the first retractor member 415 and impart a rotational force on the first retractor member 415. This rotational force may cause or force the first retractor member 415 to expand. In some examples, the actuator assembly 410 may couple or connect with the retractor assembly 405 by way of a friction fit connection, a bayonet-type connection, a secondary fastener (such as a set screw or other fastener), a quick-connect type mechanism, or other fully releasable coupling mechanisms, or a combination thereof and as described with respect to other examples.

In an example, the first retractor member 415 expands and pushes against the tissue in an outward direction and into a ring shape with the visualization channel 440 in the center. The healthcare professional may use the opening formed by the visualization channel 440 to visualize the surgical site and operate instruments within it.

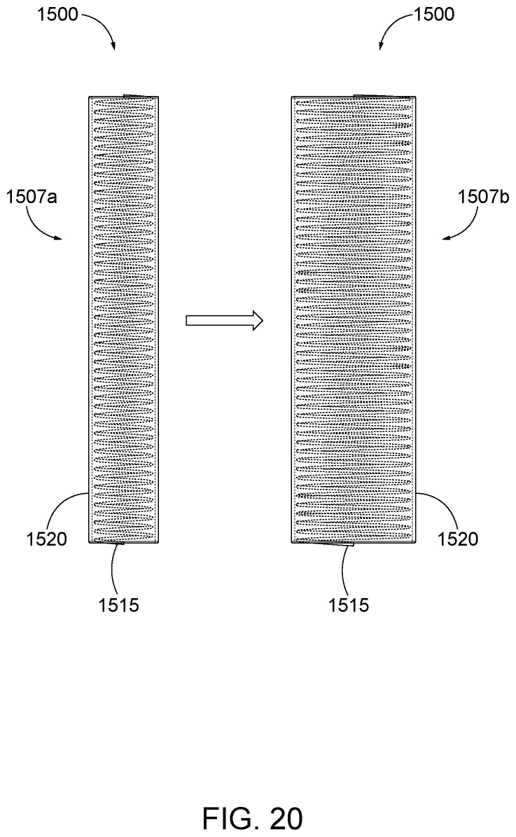

In some examples, the visualization device 1500 of FIG. 20 may be expanded similarly to the visualization device 400 of FIGS. 15-19, and 57. The expansion process of visualization device 1500 may differ in that the second retractor member 1520, such as an outer sheath, may not be removed from the visualization device 1500 positioned within the incision prior to expansion. Further, an actuator assembly similar to actuator assembly 410 of FIG. 57 may be actuated, such as by rotation, with respect to the first retractor member 1515 and the second retractor member 1520. A rotational force may then be imparted on the first retractor member 1515, which may then impart an expansion force against the second retractor member 1520. This rotational force may cause or force the first retractor member 1515 and the second retractor member 1520 to expand. In some examples, the actuator assembly may also impart a rotational force on the second retractor member 1520.

FIG. 20 is a side view of components of a visualization device 1500 in an unexpanded position 1507a and expanded position 1507b. In some examples, the components of the visualization device 1500 of FIG. 20 are similar to the visualization device 400 of FIGS. 15-19, 57. In some examples, the visualization device 1500 differs in that the second retractor member 1520, such as an outer sheath, is also expandable. In an unexpanded position, the first retractor member 1515 is positioned within the second retractor member 1520. In an expanded position, the first retractor member 1515 remains positioned within the second retractor member 1520.

In some examples, the visualization device 1500 of FIG. 20 may be expanded similarly to the visualization device 400 of FIGS. 15-19, and 57. The expansion process of visualization device 1500 may differ in that the second retractor member 1520, such as an outer sheath, may not be removed from the visualization device 1500 positioned within the incision prior to expansion. Further, an actuator assembly similar to actuator assembly 410 of FIG. 57 may be actuated, such as by rotation, with respect to the first retractor member 1515 and the second retractor member 1520. A rotational force may then be imparted on the first retractor member 1515, which may then impart an expansion force against the second retractor member 1520. This rotational force may cause or force the first retractor member 1515 and the second retractor member 1520 to expand. In some examples, the actuator assembly may also impart a rotational force on the second retractor member 1520.

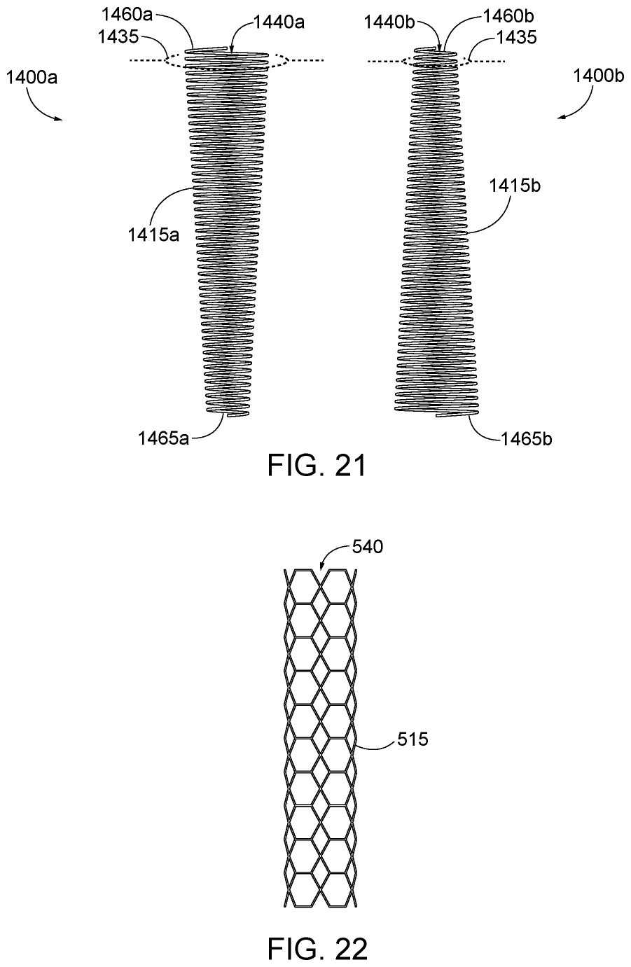

FIG. 21 is a side view of a component of a visualization device 1400a, 1400b in an expanded position as positioned in an example surgical environment. The visualization device may include a first retractor member in an expanded position with a varying width along its length between a proximal end and a distal end. In some examples, the visualization device 1400a in an expanded position has first retractor member 1415a with a proximal end 1460a that has a larger width than a width of the distal end 1465a. In this examples, the visualization channel 1440a also tapers from a larger diameter or width to a smaller diameter or width between the proximal end 1460a and the distal end 1465a. In some examples, the visualization device 1400b may be opposite that of the visualization device 1400a, with a first retractor member 1415b having a proximal end 1460b with a width that is smaller than a width of a distal end 1465b. In this examples, a visualization channel also tapers from a smaller diameter or width at the proximal end 1460a to a larger diameter or width at the distal end 1465b.

FIG. 22 shows an example of a first retractor member 515 that may be similar to the first retractor member 415 of FIGS. 15-19, and 57. A difference between the first retractor member 515 and the first retractor member 415 may be that the first retractor member 515 may be formed with a honeycomb or mesh like structure, and an internal surface of the first retractor member 515 forms a visualization channel 540. In some examples, the first retractor member 515 may be expanded in a manner similar to the first retractor member 415.

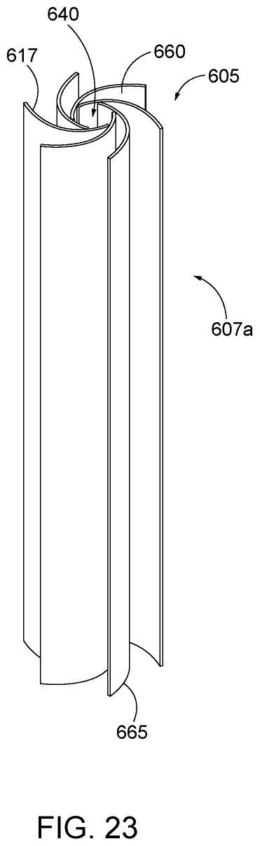

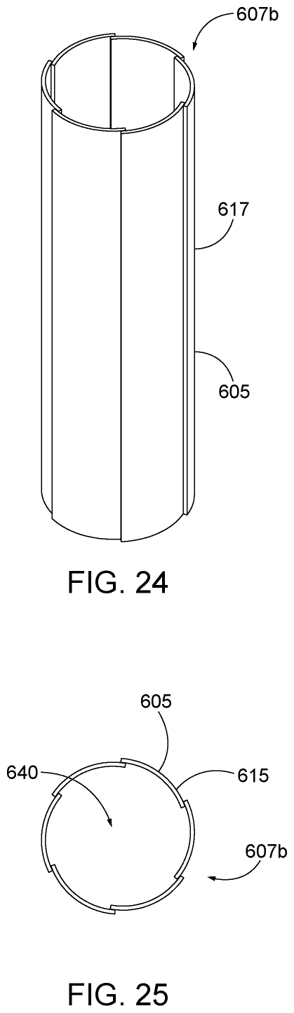

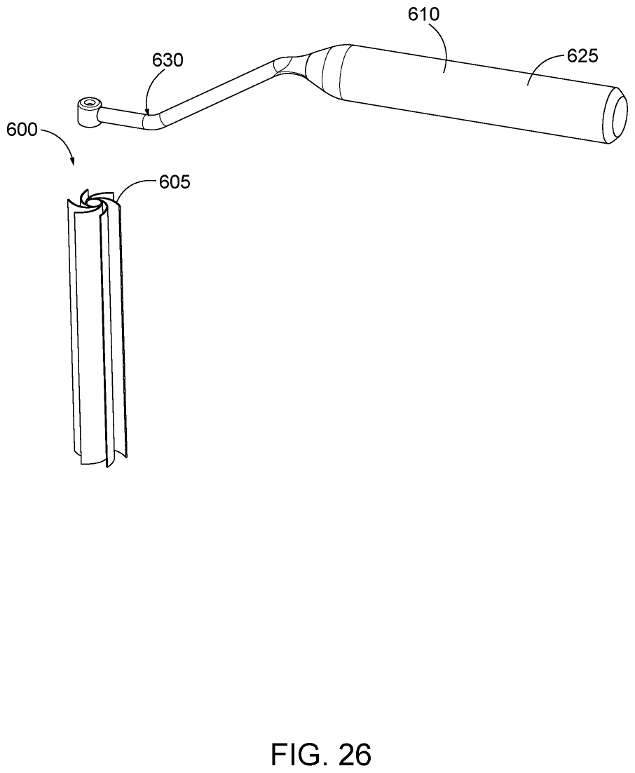

FIG. 26 shows a visualization device 600 with a retractor assembly 605 and an actuator assembly 610. In some examples, the retractor assembly 605 may be expandable about its width between a proximal end 660 and a distal end 665. A visualization channel extends in a through a central section of the retractor assembly 605 between the proximal end 660 and the distal end 665. FIG. 23 shows the retractor assembly 605 in an unexpanded position 607a. FIGS. 24-25 show the retractor assembly 605 in an expanded position 607b. As shown in FIG. 23, the retractor assembly 605 may have blades 617 that extend radially outward from a center of the retractor assembly 605. In some examples, the blades 617 are curved. In the unexpanded position 607a, the visualization channel 640 is formed by each inner portion of the blades 617 (FIG. 23). In the expanded position 607b, the blades 617 are repositioned and the visualization channel 640 may have a larger width that is generally circular (FIGS. 24-25).

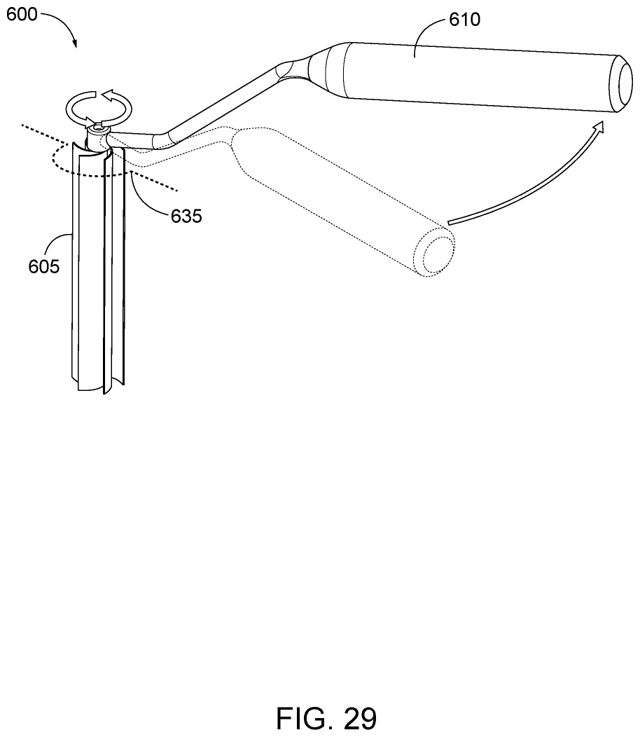

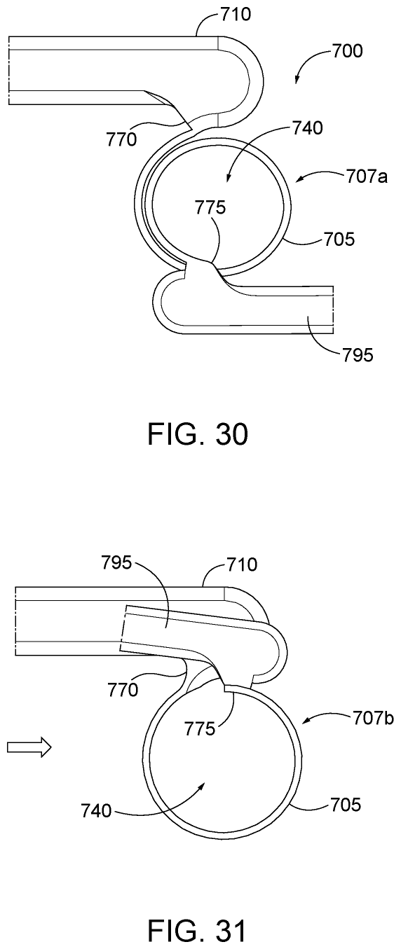

The device may further include actuator assembly 610 shown in FIGS. 26-29. The actuator assembly 610 may include a first actuator member 625 and a second actuator member 630. In some examples, the first actuator member 625 may include a handle, and the second actuator member 630 may include a loop that may couple with the retractor assembly 605. In some examples, the actuator assembly 610 may couple with the retractor assembly 605 to actuate and expand the retractor assembly 605. In some examples, the actuator assembly 610 may couple with the retractor assembly 605 in a manner similar to the retractor assembly 710 and actuator assembly 705 described with respect to FIGS. 30 and 31.

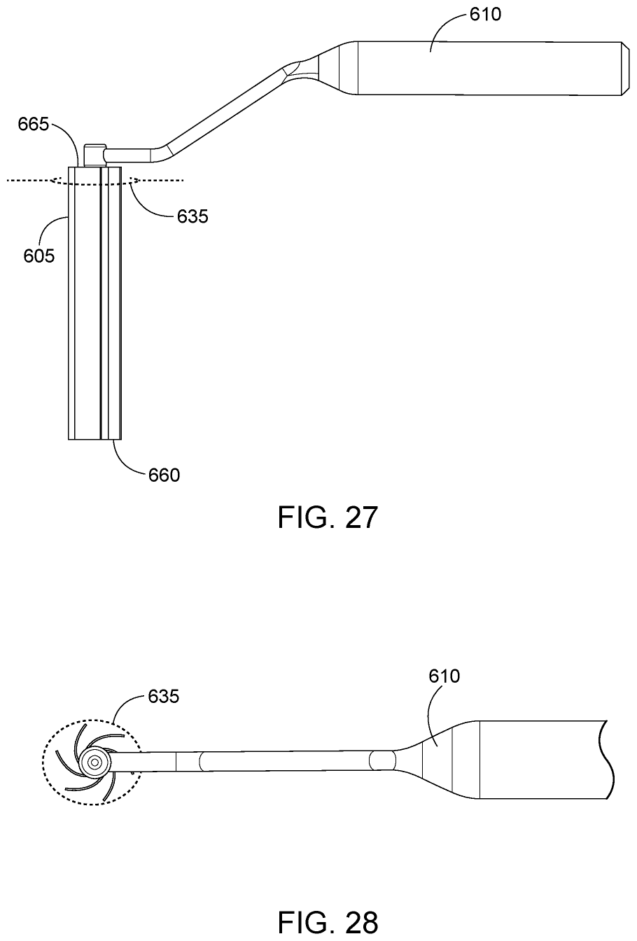

The visualization device 600 of FIGS. 27-29 may be similar the visualization device 400 of FIG. 15, with an actuator assembly 610 that may be rotationally coupled with the retractor assembly 605 to expand the retractor assembly 605 from an unexpanded position 607a (shown in FIG. 23) to an expanded position 607b (shown in FIGS. 24-25) when positioned within an incision 635. The blades 617 of the retractor assembly 605 may expand radially outward in a manner similar to an iris. As shown in FIGS. 27-29, in use, a distal end 660 of the retractor assembly 605 is placed into an incision 635. The actuator assembly 610 may then engage the retractor assembly 605, and be rotated with respect to the retractor assembly 605. As the actuator assembly 610 is rotated, the retractor assembly 605 expands as the blades 617 are moved outward in a toroidal motion, similar to an iris expanding. When transitioning from an unexpanded position 607a (FIG. 23) to an expanded position 607b (FIGS. 24-25), the blades 617 push against the tissue and displace it, and the visualization channel 640 (FIG. 25) is enlarged. Similar to the visualization device 100, in use, the health care professional is able to visualize the surgical site through the visualization channel 640, and utilize instruments through and within the visualization channel 640.

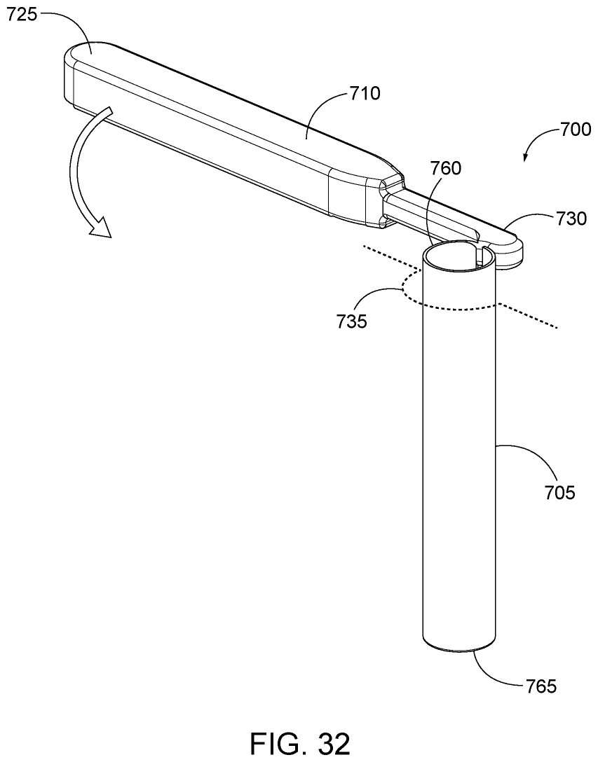

FIGS. 30-34 depict a visualization device that may be rolled. The visualization device 700 includes an actuator assembly 710 and a retractor assembly 705. FIG. 30 shows the retractor assembly 705 in an unexpanded position 707a, and FIG. 31 shows the retractor assembly in an expanded position 707b. As shown in FIG. 32, the retractor assembly 705 may be similar to an overlapping tube have a proximal end 760 and a distal end 765. In some examples, the retractor assembly 705 may be cylindrically shaped along its length between the proximal end 760 and the distal end 765 with a visualization channel 740 extending the length of the retractor assembly 705. As shown in FIG. 30, the retractor assembly 705 may have a lateral end 770 and a medial end 775 that extend between the proximal end 760 and distal end 765. As shown in FIG. 30, in an unexpanded position 707a, the medial end 775 may be rolled or positioned within the retractor assembly 705 and overlapped by the lateral end 770, similar to a scroll. In the unexpanded position, the medial end 775 is adjacent the visualization channel 740. As shown in FIG. 31, in an expanded position 707b, the medial end 775 may be positioned adjacent the lateral end 770 so that the medial end 775 and the lateral end 770 do not overlap. In this position, both the lateral end 770 and medial end 775 are positioned adjacent the visualization channel 740. The overall size of the visualization channel is larger in the expanded state 707b than in the unexpanded state 707a.

Another example of an apparatus to engage an expandable visualization device is the actuator assembly 710 shown in FIGS. 32-34. The actuator assembly may have a first actuator member 725 and a second actuator member 730. In some examples, the first actuator member 725 is a handle. In some examples, the second actuator member 730 couples with the retractor assembly 705. As shown in FIG. 30, in some examples, the second actuator member 730 is fixed or permanently attached to the retractor assembly 705 at the lateral end 770 in FIG. 30. A second handle 795 may be attached or fixed to the retractor assembly 705 at the medial end 775 with the handle 795 oriented 180 degrees away from the handle 725. Holding one handle fixed and rotating the other (or squeezing them together) may open or actuate the retractor assembly 705. The handles 725, 795 can be locked or fixed to each other to keep the retractor assembly 705 in the open position.

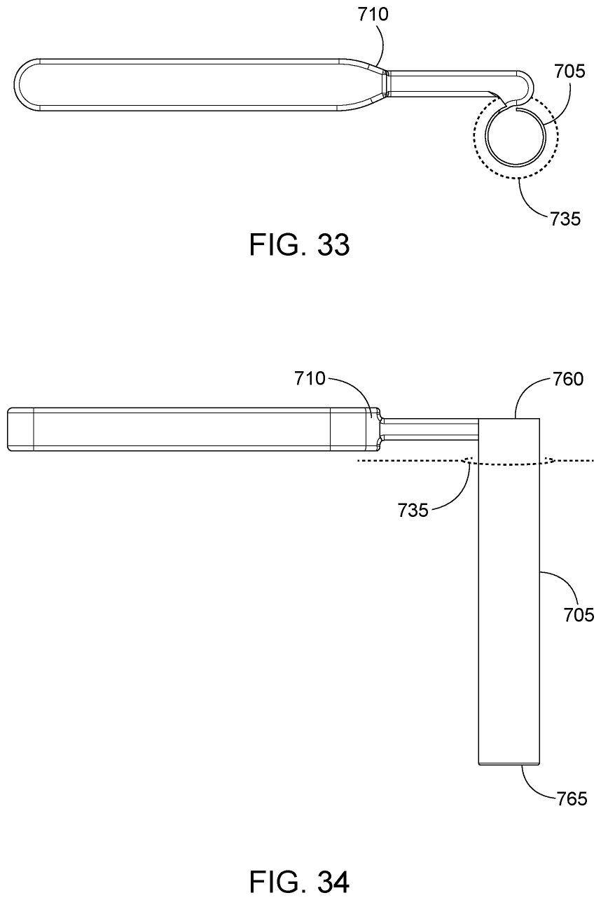

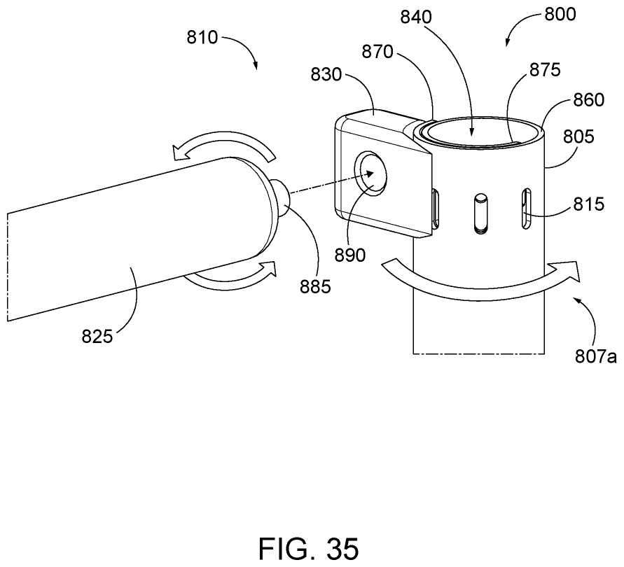

In some examples, the visualization device 800 may be similar to the visualization device 700 of FIGS. 30-34. FIG. 35 shows a visualization device 800 with a retractor assembly 805, similar to an overlapping tube, having a proximal end 860, a lateral end 870 opposite a medial end 875, and a first retractor member 815. In some examples, the first retractor member 815 may include a series of slots positioned near the proximal end of the retractor assembly 805. Similar to the retractor assembly 705 of FIG. 33, the medial end 875 may be rolled or positioned within the retractor assembly 805 and overlapped by the lateral end 880 in an unexpanded position 807a. In this unexpanded position 807a, a visualization channel 840 extends through the length of the retractor assembly 805 and is adjacent the medial end 875.

In some examples, the visualization device 800 also includes an actuator assembly 810 with a first actuator member 825 and a second actuator member 830. In some examples, the first actuator member 825 engages with a feature of the second actuator member 830. In some examples, the first actuator member 825 may rotatably engage with the second actuator member 830. The first actuator member 825 may include a protrusion or post 885 that may extend or be positioned within a cavity formed by an aperture 890 in the second actuator member 830. The actuator assembly 810 may also include features so that the engagement of the actuator assembly 810 and the retractor assembly 805 is similar to a worm drive. In some examples, the post 885 may be threaded to act as a driver of the worm drive and the second actuator member 830 may constrain the an overall width or diameter of the retractor assembly 805 or the position of the lateral edge 870 with respect to the medial edge 875 of the retractor assembly 805.

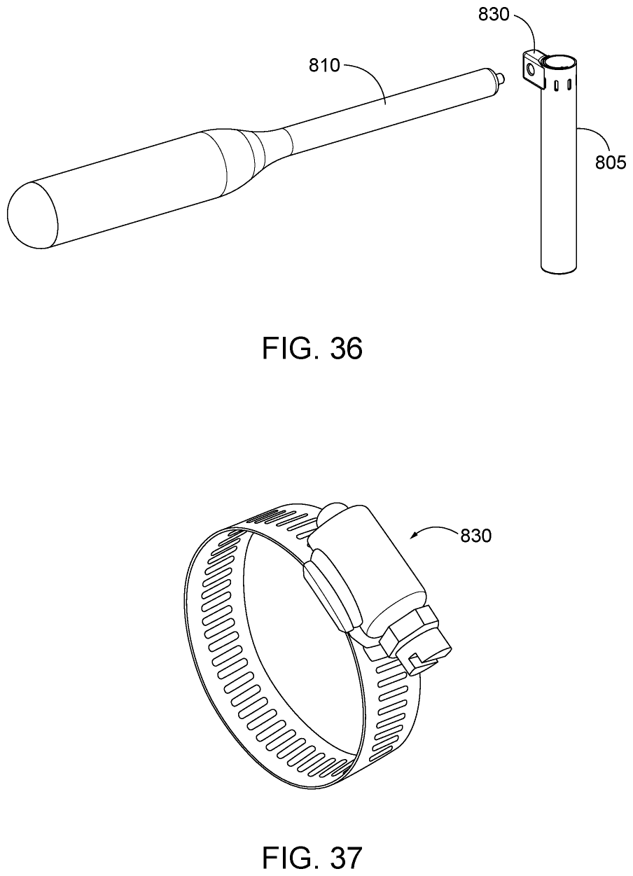

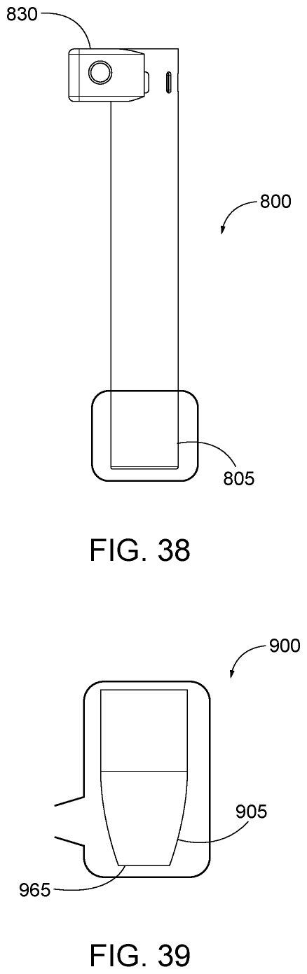

Another example of how an actuator assembly may be used to engage and expand a retractor assembly of a visualization device is shown in FIGS. 35-38. In some examples, the post 885 of the first actuator member 825 is positioned within the cavity formed by the aperture 890 in the second actuator member 830. When a user wants to expand the retractor assembly 805, the first actuator member 825 is rotated, which causes the rotation of the post 885. As the post 885 rotates, the second actuator member 830 engages with the first retractor member 815 of the retractor assembly 805 and the retractor assembly 805 expands. The expansion of the retractor assembly 805 may enlarge the visualization channel 840.

FIG. 39 shows a distal end 965 of a retractor assembly 905 of a visualization device 900. The components of the visualization device 900 of FIG. 39 may be similar to components of the visualization device 700 of FIGS. 30-34. In some examples, the distal end 965 may be tapered inward. The tapered feature of the distal end 965 may help the retractor assembly 905 to more easily be positioned adjacent the target surgical site during a surgical procedure.

The visualization device 700 of FIGS. 32-34 may be similar the visualization device 400 of FIG. 15, with an actuator assembly 710 that may be rotationally coupled with the retractor assembly 705 to expand the retractor assembly 705 from an unexpanded position 707a (shown in FIG. 32) to an expanded position 707b (shown in FIG. 31).

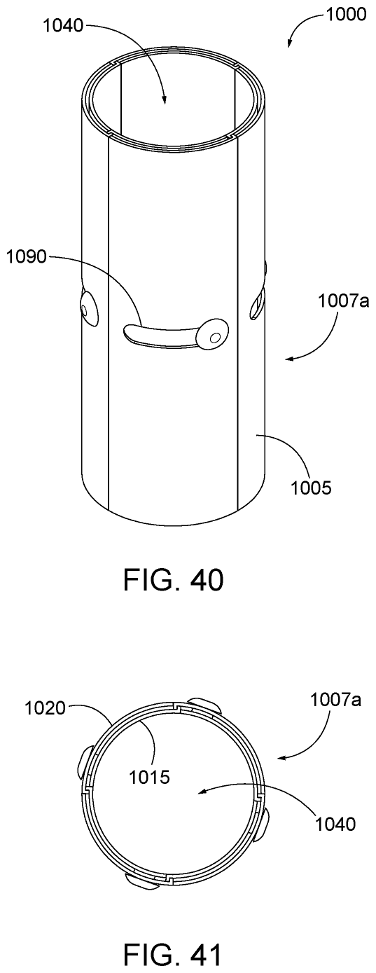

FIG. 40 depicts a visualization device 1000 including a retractor assembly 1005 in an unexpanded position 1007a. In some embodiments, the retractor assembly 1005 includes a first retractor member 1015 and a second retractor member 1020. In some examples, the first retractor member 1015 may act as an inner sheath positioned or nested within the second retractor member 1020, which may act as an outer sheath. A visualization channel 1040 may extend through a length of the retractor assembly 1005 and be formed by an inside surface of the first retractor member 1015. In some examples, the retractor assembly 105 may be made up of multiple first retractor members 1015 and second retractor members 1020.

As shown in FIG. 46, each of the first retractor members 1015 may be coupled to a corresponding second retractor member 1020 at a joint 1080. A pin 1085 may also be coupled to the first retractor member 1015. The pin 1085 may have an elongated body with a diameter and a head with a diameter that is larger than the elongated body's diameter. In some examples, the second retractor member 1020 may have an aperture or slot 1090 formed at various locations about a circumference of the retractor assembly 1005 (see FIG. 40). The slot 1090 may be sized so that the elongated body of the pin 1085 may slide within the slot 1090. In some examples, the pin 1085 adjustably couples the second retractor member 1020 to the first retractor member 1015.

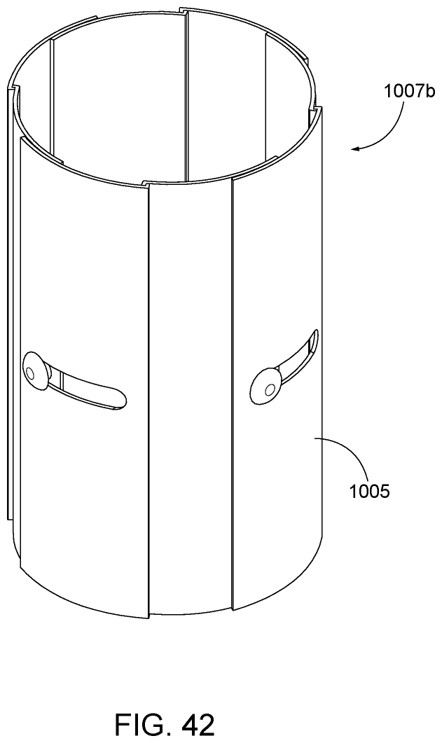

FIG. 42 shows the retractor assembly 1005 in an expanded positioned 1007b. In this position, the location of the first retractor member 1015 is adjusted with respect to the second retractor member 1020 so that a diameter of the visualization channel 1040 is increased, and the visualization channel 1040 is formed by an inner surface of the first retractor member 1015 and the inner surface of a portion of the second retractor member 1020.

In some examples, the retractor assembly 1005 is formed of a semi-flexible material, such as a sheet metal or other flexible materials that may allow the retractor assembly 1005 to bend or flex to an expanded shape.

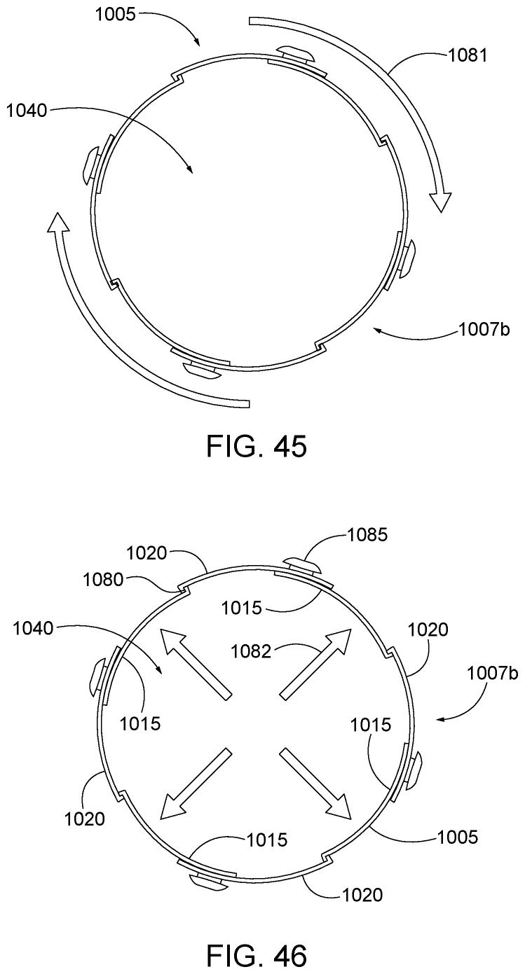

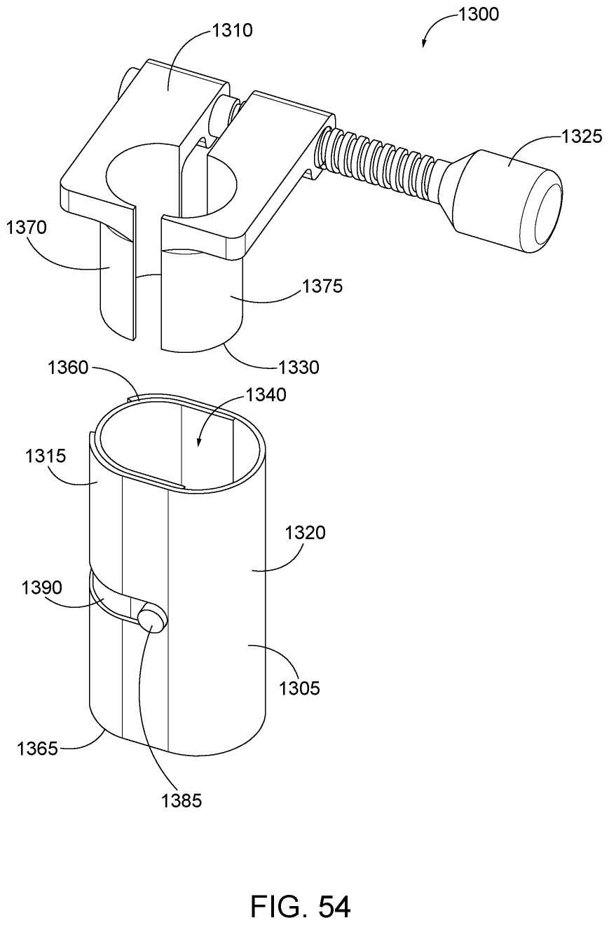

Another example of how an actuator assembly may be used to engage and expand a retractor assembly of a visualization device is shown with reference to FIGS. 40, 42, 45, and 46. In this example, once the retractor assembly 1005 is positioned within the incision 1035, the retractor assembly 1005 may be actuated by an actuator assembly (not shown) to expand the retractor assembly 1005 from an unexpanded position 1007a (FIG. 40) to an expanded position 1007b (FIG. 42). In some examples, the retractor assembly 1005 may be expanded when an actuator assembly is engaged to apply a torsional force to the second retractor member 1020, as indicated by the directional arrow 1081 in FIG. 45. In other examples, the retractor assembly 1005 may be expanded when an actuator assembly applies a force directed radially outward on the first retractor member 1015, as indicated by directional arrow 1082. In some examples, an actuator assembly similar to the actuator assemblies 110, 410, 610 of FIGS. 1, 15, 27 may be modified to expand the retractor assembly 1015. In some examples, a device similar to the actuator assembly 710 and retractor assembly 705 of FIGS. 30 and 31 may be used to expand the retractor assembly 1005. In some examples, a rolled tube or device of the actuator assembly 1010 may be inserted into or engage with the first retractor member 1015. The rolled tube or device may then be expanded, causing the first retractor member 1015 to expand. The rolled tube or device may then be removed or disconnected from the first retractor member 1015. In some examples, the actuator assembly described in FIGS. 54-56 may be used.

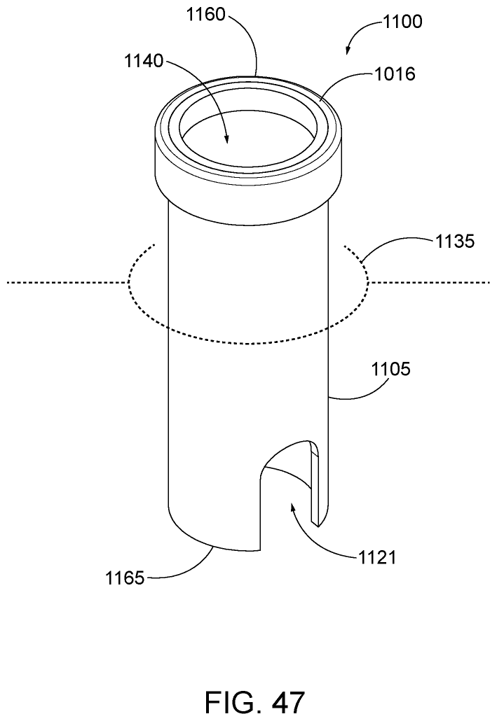

FIGS. 47-49 depict a visualization device that may be used with a light source. The visualization device 1100 may include a retractor assembly 1105 having a visualization channel 1040, a visualization aid 1016, and a tool portal 1121. In some examples, the visualization aid 1016 is positioned on a proximal end 1160 of the retractor assembly 1105. In some examples, the visualization aid 1016 may produce or transmit light into the visualization channel 1140. In some examples, the visualization aid 1016 is removably attached to the retractor assembly 1105.

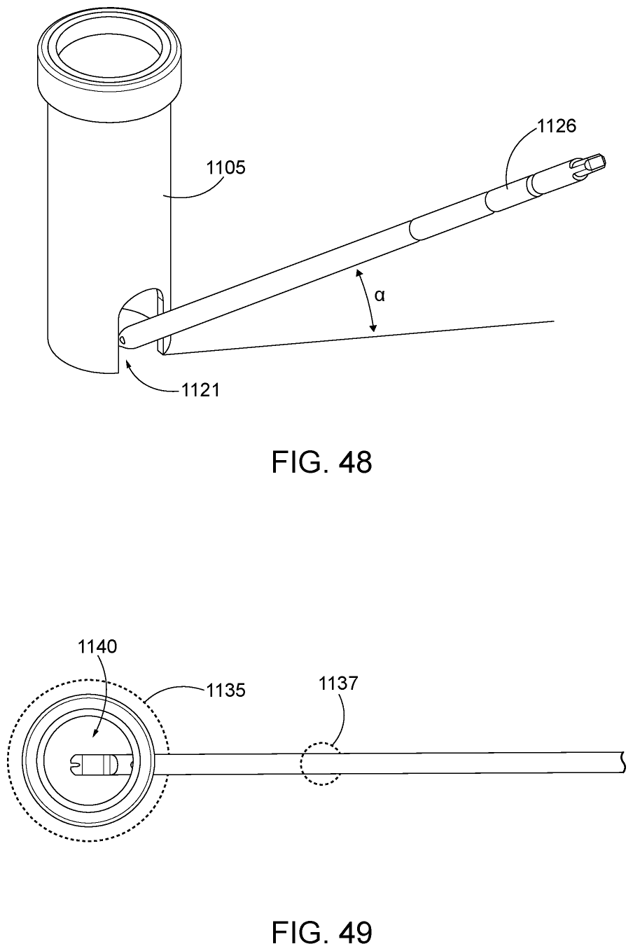

In some examples, a tool portal 1121 is formed on a distal end 1165 of the retractor assembly 1105. The tool portal 1121 may be formed as a cutout shape that extends from the distal end 1165 and partially along a length of the retractor assembly 1105 towards the proximal end 1160. In some examples, the tool portal 1121 may be sized to allow a surgical tool or instrument 1126 (see FIG. 48), such as a chisel or other surgical instruments, to access the visualization channel 1140 through the tool portal 1121.

Another example method of actuating a visualization device is shown with reference to FIGS. 47-49. In an example, an incision 1135 may be made in the posterior cervical spine, and manual dissection is performed down to the surgical area. With the tool portal 1121 of the visualization device 1100 facing inferiorly, the retractor assembly 1105 may be inserted through the incision 1135 until the distal end 1165 of the retractor assembly 1105 contacts the targeted surgical site. This may help dilate the incision 1135 and provide a user with a direct visualization of the surgical site via the visualization channel 1140. In some examples, the visualization aid 1016 is engaged to improve visibility of the visualization channel 1140 and the surgical site. In some examples, the visualization aid is a light or device that provides additional light.

As shown in FIG. 49, a second incision 1137 may be made inferior to the retractor assembly 1105. The surgical instrument 1126 may then be inserted into incision 1137 at an angle α with respect to the retractor assembly 1105. In some examples, the angle α is approximately 30 degrees from a plane normal to the visualization channel 1140.

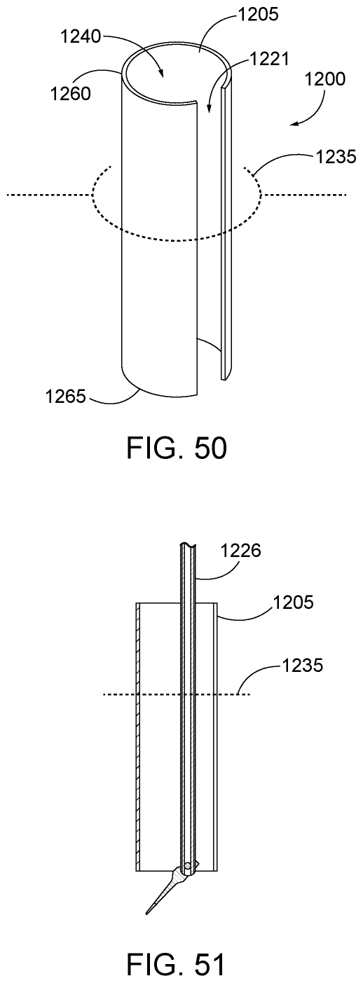

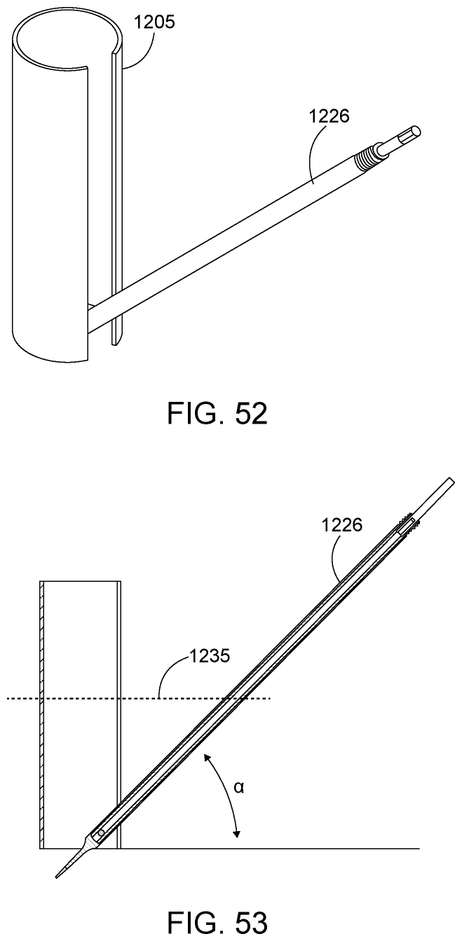

The visualization device 1200 of FIGS. 50-53 may be similar to the visualization device 1100 of FIGS. 47-49. A difference may be that a tool portal 1221 may extend a length of the retractor assembly 1205, from a proximal end 1260 to a distal end 1265. The retractor assembly 1205 may be formed as a sleeve with an adjustable diameter.

Another example method of actuating a visualization device is shown with reference to FIGS. 50-53. Similar to the method using visualization device 1100 of FIGS. 47-49, an incision 1235 may be made in the posterior cervical spine, and manual dissection is performed down to the surgical area. With the tool portal 1221 of the visualization device 1200 facing inferiorly, the retractor assembly 1205 is inserted through the incision 1235 until the distal end 1265 of the retractor assembly 1205 contacts the targeted surgical site. A surgical tool 1226, such as a pivoting access chisel, may be inserted vertically down from the proximal end 1260 to the distal end 1265 to access the target surgical site, such as a cervical spine joint, at the proper angle. The surgical tool 1226 may pivot at the spinal joint to a straight position. The tool portal 1221 may allow the surgical tool 1226 to reach a desired angle α, as shown in FIG. 53. Additional surgical instruments may be made to allow full extension of the surgical tool 1226. In some examples, the incision 1235 may be extended if necessary based upon a desired position of the surgical tool 1226.

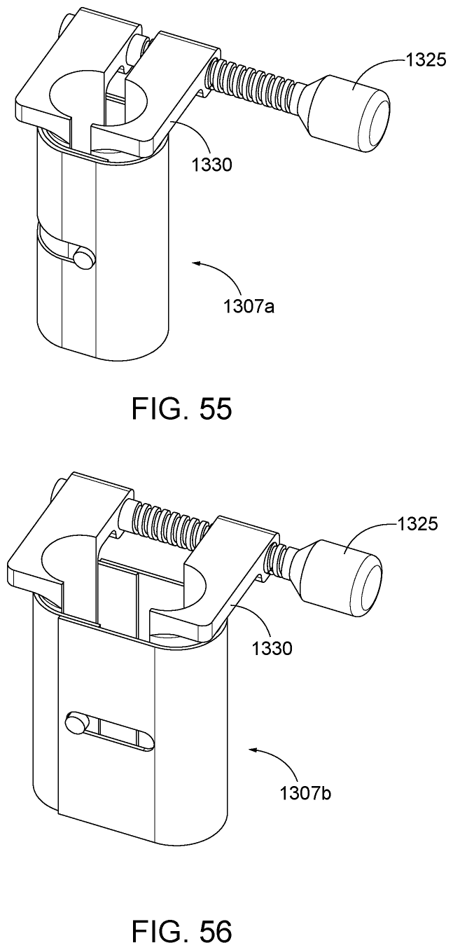

FIGS. 54-56 depicts a visualization device with an adjustable channel. The visualization device 1300 may include a retractor assembly 1305 and an actuator assembly 1310. The retractor assembly 1305 may have a proximal end 1360 opposite a distal end 1365, and include a first retractor member 1315, a second retractor member 1320, and a visualization channel 1340 that extends through a central portion of the retractor assembly 1305 from the proximal end 1360 to the distal end 1365. In some examples, the position of the first retractor member 1315 with respect to the second retractor member 1320 may be adjustable so that the overall width of the visualization channel 1340 may be enlarged. In some examples, a pin 1385, similar to pin 1085 of FIG. 46, may be coupled to the first retractor member 1315, with a portion of the pin 1385 positioned within a slot 1390 of the second retractor member 1320, similar to slot 1090 of FIG. 40. As shown in FIG. 55, the pin 1385 is adjacent a first end of the slot 1390 in unexpanded position 1307a. As shown in FIG. 56, the pin 1385 is adjacent an opposite, second end of the slot 1390 in an expanded position 1307b. In some examples, the first retractor member 1315 and the second retractor member 1320 may be formed from a flexible material, such as thin sheet metal or a polymer.

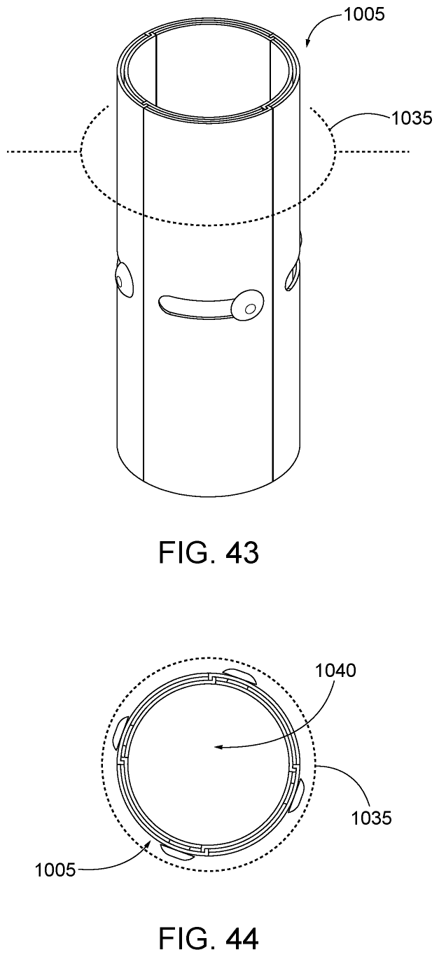

Another example of an apparatus to engage an expandable visualization device is the actuator assembly 1310 shown in FIGS. 54-56. The actuator assembly 1310 may include a first actuator member 1325 and a second actuator member 1330. In some examples, the first actuator member 1325 may include a threaded rod that can be actuated by a user. The second actuator member 1330 may include a first actuator paddle 1370 and a second actuator paddle 1375. The second actuator member 1330 may have a first end coupled to the first actuator member 1325 and a second end that may engage with the retractor assembly 1305. In an example, the first actuator paddle 1370 is coupled to the first actuator member 1325 such that a rotation of the first actuator member 1325 is not transmitted to the first actuator paddle 1370. In an example, the second actuator paddle 1375 is coupled to the first actuator member 1325 such that a rotation of the first actuator member 1325 adjusts the linear position of the second actuator paddle 1375 with respect to the first actuator paddle 1370. The visualization device 1300 may be actuated similar to the visualization devices 1000 of FIGS. 40-46. In an example, after an incision is made, the distal end 1365 of the retractor assembly 1305 is inserted into the incision and positioned adjacent the target surgical area. The second actuator member 1330 is inserted into the visualization channel 1340 formed by the first retractor member 1315 and the second retractor member 1320. The first actuator paddle 1370 may be positioned adjacent an inside surface of the first retractor member 1315, and the second actuator paddle 1375 may be positioned adjacent an inside surface of the second retractor member 1320. A user, such as a healthcare professional, may then engage the first actuator member 1325 by rotating it, and cause the linear movement of the second actuator paddle 1375 away from the first actuator paddle 1370. The pin 1385 and slot 1390 align the first retractor member 1315 and the second retractor member 1320 during the expansion process. Once the desired expansion of the retractor assembly 1305 is achieved, a user may access the surgical site via the visualization channel 1340.

All relative and directional references (including: upper, lower, upward, downward, left, right, leftward, rightward, top, bottom, side, above, below, front, middle, back, vertical, horizontal, and so forth) are given by way of example to aid the reader's understanding of the particular embodiments described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other unless specifically set forth in the claims.

Those skilled in the art will appreciate that the presently disclosed embodiments teach by way of example and not by limitation. Therefore, the matter contained in the above description or shown in the accompanying drawings should be interpreted as illustrative and not in a limiting sense. Thus, it is intended that the scope of the present disclosure should not be limited by the particular embodiments described above. Changes in detail or structure may be made without departing from the spirit of the invention as defined in the appended claims. In methodologies directly or indirectly set forth herein, various steps and operations are described in one possible order of operation, but those skilled in the art will recognize that steps and operations may be rearranged, replaced, or eliminated without necessarily departing from the spirit and scope of the present invention as claimed below.

What is claimed is:

1. A visualization device comprising:
a retractor assembly having a visualization channel, the retractor assembly comprising a first retractor member and a second retractor member, and the retractor assembly configured to be coupled to an actuator assembly configured to actuate the retractor assembly.

2. (canceled)

3. The device of claim 2, wherein the first retractor member is expandable.

4. The device of claim 2, wherein the first retractor member is expanded and configured to cause the second retractor member to expand.

5. The device of claim 2, wherein the first retractor member is radially expandable.

6. The device of claim 2, wherein the first retractor member is configured to move with respect to the second retractor member or the second retractor member is configured to move with respect to the first retractor member.

7. (canceled)

8. The device of claim 2, wherein the first retractor member and the second retractor member are configured to move with respect to each other.

9. The device of claim 1, wherein the first retractor member comprises a mesh structure formed into an expandable spring or spring-like configuration having looped spring coils stacked between a proximal end and a distal end, and the visualization channel is formed by an inside surface of the coil springs.

10. The device of claim 1, wherein the first retractor member is positioned within the second retractor member.

11. The device of claim 1, wherein the second retractor member is a sheath.

12. The device of claim 1, wherein the first retractor member forms the visualization channel.

13. The device of claim 12, wherein a surface of the first retractor member forms the visualization channel.

14. The device of claim 1, wherein the first retractor member comprises radially expanding blades.

15. The device of claim 1 further comprising the actuator assembly, wherein the actuator assembly is configured to be rotated with respect to the retractor assembly.

16. The device of claim 1, wherein the retractor assembly is toroidally expandable.

17. (canceled)

18. The device of claim 1, wherein the visualization device is configured for insertion into a surgical site and the surgical site is in the cervical spine.

19. The device of claim 1, wherein a distal end of the retractor assembly is tapered.

20. The device of claim 1, wherein the retractor assembly has an adjustable diameter.

21. A visualization device configured for a surgical site, the device comprising:
a retractor assembly comprising a visualization channel extending from a proximal end to a distal end of the retractor assembly; and a tool portal adjacent the distal end of the retractor assembly.

22. (canceled)

23. (canceled)

24. (canceled)

25. The device of claim 21, wherein the retractor assembly comprises a sleeve with an adjustable diameter.

26. A method of using a visualization device in a surgical site comprising:
inserting at least a portion of a retractor assembly into an incision and positioning adjacent the surgical site;
engaging an actuator assembly with the retractor assembly;
expanding the retractor assembly; and
expanding a visualization channel of the retractor assembly extending from a proximal end to a distal end of the retractor assembly.

27-35. (canceled)
